(19) United States
(12) Patent Application Publication
(10) Pub. No.: US 2022/0226239 A1
(43) Pub. Date: Jul. 21, 2022

(54) POLYMERIC MICELLE COMPLEXES, FORMULATIONS, AND USES THEREOF

(71) Applicant: ARAVASC INC., Sunnyvale, CA (US)

(72) Inventors: Narmada SHENOY, Sunnyvale, CA (US); Suganya SELVARAJAH, San Francisco, CA (US)

(73) Assignee: ARAVASC INC., Sunnyvale, CA (US)

(21) Appl. No.: 17/616,610

(22) PCT Filed: Jun. 4, 2020
(86) PCT No.: PCT/US2020/036082

§ 371 Date: Dec. 3, 2021

Related U.S. Application Data
(60) Provisional application No. 62/857,212, filed on Jun. 4, 2019.

(51) Int. Cl. A61K 9/107; A61K 31/7084; A61K 47/34

(52) U.S. Cl. CPC A61K 9/1075; A61K 47/34; A61K 31/7084

(57) ABSTRACT

The disclosure provides compositions of polymeric micelle complexes, as well as methods for preparing such compositions. Such compositions are suitable for pharmaceutical delivery of one or more ionic agents to cell interior, and can be used in therapy and/or diagnosis, for example, for treating cancer as well as other diseases depending on the ionic agent.

### CROSS REFERENCE TO RELATED APPLICATIONS

This application claims priority to U.S. Provisional Application Ser. No. 62/857,212, filed Jun. 4, 2019, which is hereby incorporated herein by reference in its entirety.

### FIELD

The present invention relates to compositions containing one or more ionic agents in a polymeric micelle for delivery to the cell interior, and methods of using such compositions in therapy and/or diagnosis, for example, for treating diseases such as cancer.

### BACKGROUND

It is generally desirable to provide pharmaceutical actives in formulations targeted to the disease site in order to permit lower dosing, reduce side effects, and/or to improve patient compliance. Ibis may be particularly true in the case of drugs that tend to have unpleasant side effects and/or undesired degradation, such as certain anti-cancer agents.

Polymer-therapeutics are gaining wide acceptance as drug delivery systems. Polymer-therapeutics involve the use of polymeric systems to enhance the drug's circulation half-life and to reduce its toxicity. Polymeric micelles are formed by spontaneous self assembly of amphiphilic copolymers. Amphiphilic copolymers are composed of hydrophobic and hydrophilic segments, arranged in either block or graft architecture. Generally speaking, the amphipilic copolymers in aqueous medium undergo micellization by aggregation of their hydrophobic domains.

Many known polymeric micellar systems are designed to accumulate at the tumor site passively, due to the size of the delivery vehicle, through the leaky vasculature at the tumor site. It is widely recognized that polymeric micellar systems are capable of encapsulating water insoluble agents in the inner hydrophobic core by hydrophobic interactions. However, classical polymeric micelles exhibit poor encapsulation efficiency for water soluble agents. In addition, desire for development of a pharmaceutical preparation which can maintain, if possible, a polymer micelle form under physiological environment over longer period of time shall still be present.

Therefore, there exists a great deal of interest enhancing the loading efficiency and stability in polymeric micellar systems.

### BRIEF SUMMARY

The present disclosure meets the unmet needs described above by providing compositions and kits comprising one or more ionic agents complexed with a polymeric micelle. The polymeric micelle complex addresses the loading efficiency issue as well as any potential cytotoxicity and stability problems associated with the ionic agent used for therapy.

In some embodiments, the present disclosure provides a composition comprising a polymeric micelle complex comprising:
i) a plurality of block copolymers, wherein each block copolymer comprises at least a pentablock represented by formula (1) of—[A]-[B]—[C]-[D]-[E]—,
   wherein the repeating units of blocks [A] and [E] are each independently comprising a pendant moiety carrying a first charge, and
   wherein blocks [B], [C] and [D] are independently poly(alkylene oxide);
wherein the plurality of pentablock copolymers are arranged into a micelle with an interior hydrophobic core and an exterior hydrophilic layer; and
ii) one or more ionic agents comprising a first ionic agent, wherein the first ionic agent carries a second charge that is opposite to the first charge of the pendant moiety, wherein the first ionic agent complexes with at least a portion of the pendant moieties in the polymeric micelle.

In some embodiments, the ionic agent is a drug molecule, such as, Floxuridine, Fluorouracil, Azathioprine, Thiopurines, Fludarabine, Gemcitabine, Cytarabine, Methotrexate, Pemetrexed and Paracetamol, or a derivative and/or metabolite thereof. In some embodiments, the ionic agent is 5-fluom-2′-deoxyuridine-5′-O-monophosphate (FdUMP). In some embodiments, the ionic agent is cyclic guanosine monophosphate-adenosine monophosphate (cGAMP). In some embodiments, the ionic agent is a nucleic acid. The nucleic acid can be DNA or RNA. For example, the ionic agent can be mRNA. In some embodiments, the one or more ionic agents are any combination of FdUMP, cGAMP, and mRNA.

Other aspects of the present disclosure relate to a pharmaceutical composition containing a composition of any of the embodiments described herein, and a pharmaceutically acceptable carrier.

Other aspects of the present disclosure relate to a kit containing a composition of any of the embodiments described herein for use in any of the methods described herein.

Other aspects of the present disclosure relate to a method for delivering one or more ionic agents to a subject in need thereof, by administering to the subject a composition of any of the embodiments described herein. Also provided is a method for delivering one or more ionic agents to a cell interior of a subject in need thereof, by administering to the subject a composition of any of the embodiments described herein. In some embodiments, the composition is administered orally, topically, dermally, nasally, intravenously, intramuscularly, intraperitoneally, intracerobrospinally, intacranially, intraspinally, subcutaneously, intraarticularly, intrasynovialy, or intrathecally.

Other aspects of the present disclosure relate to a method for treating cancer, and/or other diseases such as liver diseases, in a subject in need thereof, by administering to the subject a composition of any of the embodiments described herein.

In further aspects, provided are method of preparing a composition of any of the embodiments described herein. In some embodiments, the composition is a polymeric micelle complex. In some embodiments, the composition is a pharmaceutical composition comprising such a polymeric micelle complex.

### DESCRIPTION OF THE DRAWINGS

The present application can be best understood by reference to the following description taken in conjunction with the accompanying figures included in the specification.

FIG. 1 illustrates the comparison of dialysis results between 5-fluoro-2′-deoxyuridine-5′-O-monophosphate (FdUMP) complexed with the pentablock copolymer at N/P of 100 and free FdUMP. FdUMP in Formulation A1 at N/P ratio of 100 showed almost 100% complexation. Upon dialysis for 2 days, <5% free FdUMP was detected. Quantitation was done by HPLC:C18 column at 270 nm; MP:Water:CH₃CN:MeOH (60:20:20).

FIG. 2 is a graph of NMR Spectrum of the pentablock copolymer.

FIG. 3 depicts the plasma concentration profile of FdUMP demonstrating that FdUMP plasma exposure is prolonged as Formulation A (t½=53 hr IV and 116 hr SC) (vs short half-life<10 mins for fluorouracil (5-FU) and FdUMP). Plasma levels of FdUMP following single IV and single SC bolus in rats (n=3) at 1.25 mg/kg over 48 hours. FdUMP levels were measured by an LC/MS/MS method. API-4000Qtrap Mass Spectrometer. ESI negative, MRM Scan at 327.06; Shimadzu HPLC/CTC with CL-S2 column (2.1×100 mm, 5 μm); MPA:MPAB: 2% Formic acid in 5 mM NH₄Ac:100% 1% Formic acid in CH₃CN; Linearity: 1-2000 ng/ml FdUMP; LOQ=1 ng/ml.

FIG. 4 depicts the plasma concentration profile of FdUMP demonstrating that FdUMP plasma exposure is prolonged as Formulation B. However complexation is non-optimal and much of the drug is seen within 10 mins. Plasma levels of FdUMP following single IV bolus in rats (n=3) at 5 mg/kg over 72 hours. FdUMP levels were measured by an LC/MS/MS method. API-4000Qtrap Mass Spectrometer. ESI negative, MRM Scan at 327.06; Shimadzu HPLC/CTC with CL-S2 column (2.1.×100 mm, 5 μm); MPA:MPAB: 2% Formic acid in 5 mM NH₄Ac:100% 1% Formic acid in CH₃CN; Linearity: 1-2000 ng/ml FdUMP; LOQ=1 ng/ml.

FIG. 5 depicts the plasma concentration profile of FdUMP demonstrating that FdUMP with no polymer as Formulation C has a short half-life of <10 mins. Plasma levels of FdUMP following single IV bolus in rats (n=3) at 1 mg/kg over 24 hours. FdUMP levels were measured by an LC/MS/MS method. API-4000Qtrap Mass Spectrometer. ESI negative, MRM Scan at 327.06; Shimadzu HPLC/CTC with CL-S2 column (2.1×100 mm, 5 μm); MPA:MPAB: 2% Formic acid in 5 mM NH₄Ac:100% 1% Formic acid in CH₃CN; Linearity: 1-2000 ng/ml FdUMP; LOQ=1 ng/ml.

FIG. 6 depicts the plasma concentration profile of indocyanine green (ICG) as Formulation D of ICG (250 μg/ml) complexed with PBC (20 mg/ml). Plasma levels of ICG following single IV bolus in rats (n=3) at 1.25 mg/kg over 48 hours. ICG levels were measured by a Tecan, 96-well plate fluorescent plate reader method. LOQ=1 ng/ml. Complexation with ICG is not optimal however a prolonged exposure of ICG until 48 hours is observed.

FIG. 7 depicts an agarose gel analysis demonstrating the formation of polymer complexes with mRNA. Formation of mRNA-PBC complexes was measured by loading of 20 μL of sample at the center of a 1% agarose gel and run for 30 minutes. mRNA in Formulation F samples migrate towards the positive pole (+Ve), while mRNA-PBC in Formulations E, E1, and E2 samples migrate towards the negative pole (−Ve). No signal is detected for Formulation G. The agarose gel was visualized with a LI-COR Oddissey fluorescence gel imager.

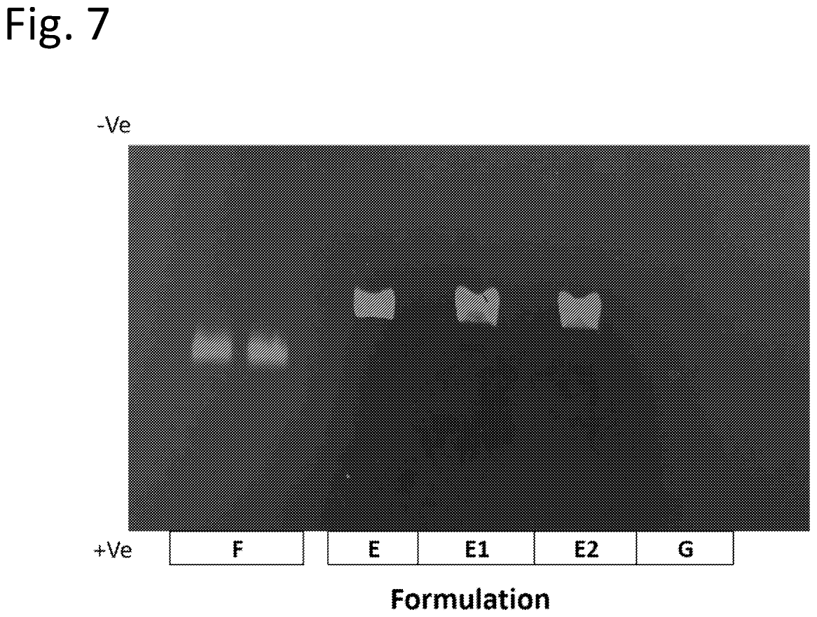

### DETAILED DESCRIPTION

The present disclosure is based on the inventors' discovery that certain micelle compositions are effective at complexing with one or more ionic agents and delivering the ionic agent to cell interior.

Unless defined otherwise, all scientific and technical terms are understood to have the same meaning as commonly used in the art to which they pertain. For the purpose of the present disclosure, the following terms are defined.

The term “about” as used herein refers to the usual error range for the respective value readily known to the skilled person in this technical field. Reference to “about” a value or parameter herein includes (and describes) embodiments that are directed to that value or parameter per se. For example, “about x” includes and describes “x” per se. In some embodiments, the term “about” when used in association with a measurement, or used to modify a value, a unit, a constant, or a range of values, refers to variations of +/−2%.

As used herein and in the appended claims, the singular forms “a,” “an,” and “the” include plural reference unless the context clearly indicates otherwise.

### Compositions

Provided herein are compositions comprising a polymeric micelle complex comprising (1) a plurality of block copolymers and (2) at least one ionic agent.

Polymeric Micelles

A polymeric micelle complex as described therein comprises a plurality of block copolymers. In some embodiments, each block copolymer comprises at least a pentablock represented by formula (I) of—[A]-[B]—[C]-[D]-[E]—, wherein the repeating units of blocks [A] and [E] each independently comprise a pendant moiety carrying a first charge, and wherein blocks [B], [C] and [D] are independently poly(alkylene oxide), so that the plurality of pentablock copolymers are arranged into a micelle with an interior hydrophobic core and an exterior hydrophilic layer.

Block copolymers of the present disclosure can include a hydrophilic and a hydrophobic segment and are able to form polymeric micelles having a core derived from the hydrophobic parts and a shell from the hydrophilic parts. In some embodiments, they exhibit pH-sensitive behavior, good water solubility and capability of thermoreversible gelation.

In some embodiments, block copolymers each block copolymer comprises at least a pentablock represented by formula (I) of—[A]-[B]—[C]-[D]-[E]—, wherein the blocks [A] and [E] each independently have a structure:

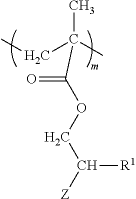

wherein R¹ is selected from the group consisting of a hydrogen and C₁₋₆ alkyl;

Z is selected from the group consisting of NR²R³, P(OR⁴)₃, SR⁵

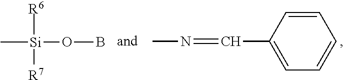

wherein R² and R³ are independently H, C₁₋₆, alkyl, or 1-mer to 28-mer oligonucleotide in which one or more of its natural phosphate backbone linkages are replaced with triazole linkages, or R² and R³ together with the nitrogen form a cyclic amine; R⁴ is C₁₋₆ alkyl; R⁵ is tri(C₁₋₆alkyl) silyl; and B is C₁₋₆ alkyl; and

m is an integer ranging from 1 to 5000.

In some embodiments, the pendant moieties of blocks [A] and [E] are cationic. In some embodiments, the pendant moieties of blocks [A] and [E] are anionic. In some embodiments, the pendant moieties of blocks [A] and [E] are amphiphilic.

In some embodiments, R² and R³ are the same or different C₁₋₆ alkyl, e.g, ethyl. In some embodiments, R² and R³ together with the nitrogen form a cyclic amine, such as pyrrolidine, piperidine, morpholine, and piperazine. In some embodiments, at least one of R² and R³ is 1-mer to 28-mer oligonucleotide in which one or more of its natural phosphate backbone linkages are replaced with triazole linkages.

In some embodiments, the alkylene oxide unit of the blocks [B] and [D] are unsubstituted and unbranched, and the alkylene oxide unit of the block [C] is substituted or branched. In some embodiments, the blocks [B] and [D] are the same

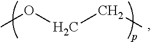

wherein p is an integer ranging from 30 to 20,000. In some embodiments, the blocks [B] and [D] are the same

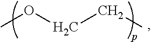

wherein p is about 100.

In some embodiments, the block [C] is

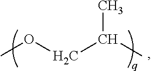

wherein p is an integer ranging from 30 to 20,000. In some embodiments, the block [C] is

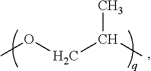

wherein q about 65.

In some embodiments, m is about 13.

In some embodiments, the block copolymers are the ones described in U.S. Pat. No. 7,217,776. In some embodiments, the block copolymers are synthesized by polymerization of a tertiary amine methacrylate with a poly(ethylene oxide)-b-poly (propylene oxide)-b-poly(ethylene oxide) (Pluronic®), including low molecular weight or high molecular weight varieties of said compounds. In certain embodiments, the block copolymers are the one synthesized in Example 2 of U.S. Pat. No. 7,217,776 having the following structure:

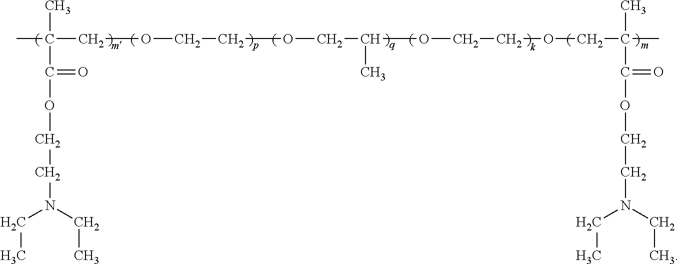

It can be prepared using N,N—(diethyl amino) ethyl methacrylate (DEAEM) as the monomer, disubstituted potassium salt of poly(ethylene oxide)-b-poly(propylene oxide)-b-poly(ethylene oxide) (Pluronic® F127. Mₙ, =12,600, 70% w/w PEG) (Sigma-Aldrich Co St. Louis, Mo.) as the polymerization initiator, and tetrahydrofuran (THF) as the solvent.

In some embodiments, the polymeric micelle complex of the present disclosure have an average particle size that ranges from about 0.01 microns in diameter to about 0.5 microns in diameter. In certain embodiments, the polymeric micelle complex of the present disclosure have an average particle size of about 0.01 microns in diameter, about 0.02 microns in diameter, about 0.03 microns in diameter, about 0.04 microns in diameter, about 0.05 microns in diameter, about 0.06 microns in diameter, about 0.07 microns in diameter, about 0.08 microns in diameter, about 0.09 microns in diameter, about 0.1 microns in diameter, about 0.15 microns in diameter, about 0.2 microns in diameter, about 0.25 microns in diameter, about 0.3 microns in diameter, about 0.35 microns in diameter, about 0.4 microns in diameter, about 0.45 microns in diameter, or about 0.5 microns in diameter. In some embodiments, the polymeric micelle complex of the present disclosure have an average particle size of about 0.025 microns to about 0.25 microns. In some embodiments, the polymeric micelle complex of the present disclosure have an average particle size of about 0.025 microns to about 0.05 microns, about 0.05 microns to about 0.1 microns, about 0.1 microns to about 0.2 microns, about 0.2 microns to about 0.3 microns, about 0.3 microns to about 0.4 microns, or about 0.4 microns to about 0.5 microns.

Ionic Agents

Other aspects of the present disclosure relate to compositions containing one or more ionic agents complexed with the polymeric micelle of the present disclosure. In some embodiments, the composition comprises an ionic agent. In some embodiments, the composition comprises two, three or four ionic agents. Any suitable ionic agents known in the art may be used.

In some embodiments, the ionic agent is a therapeutic molecule, such as, Floxuridine, Fluorouracil, Azathioprine, Thiopurines, Fludarabine, Gemcitabine, Cytarabine, Methotrexate, Pemetrexed and Paracetamol. In some embodiments, the ionic agent is a derivative and/or metabolite of the therapeutic molecule. The derivatives can be derived from those small molecule drugs by adding 1, 2 or 3 functional groups, such as phosphate, sulfate, and carboxylate.

In some embodiments, the ionic agent is 5-fluoro-2′-deoxyuridine-5′-O-monophosphate (FdUMP). 5-fluorouracil (5FU), a fluoropyrimidine, is the mainstay of a broad range of cancer treatments. Since their discovery over 5 decades ago, fluoropyrimidines such as 5FU continue to be a key component of systemic single agent or combination chemotherapy, in the treatment of colorectal and other gastrointestinal (GI) cancers, breast cancer, and head & neck cancer. It provides a significant survival benefit in colon cancer (CRC). For cytotoxic activity, 5FU requires cellular uptake and intracellular metabolic activation. Its antineoplastic effects are caused by inhibition of the nucleotide synthetic enzyme, thymidylate synthase (TS) and rapid mis-incorporation of its fluoro-metabolites into RNA and DNA resulting in DNA damage and cell death in tumor cells. Despite its long-proven efficacy, 5FU has several key drawbacks, including degradation (>80%) by dihydropyrimidine dehydrogenase (DPD) and toxic catabolites, sub-optimal Efficacy and short plasma half-life.

By utilizing a temperature and pH responsive polymeric micelle as described herein, one or more ionic agents can be delivered intracellularly. In some aspects, the disclosure provides a method of delivering one or more ionic agents such as 5-fluoro-2′-deoxyuridine-5-O-monophosphate (FdUMP), that is, the monophosphate of floxuridine to a cell interior. Without wishing to be bound by any theory, it is believed that such method of delivery enhances cellular uptake and facilitates efficient payload release in the cytoplasm.

The polymeric micelle complex of the present disclosure can be used to leverage the proven efficacy of fluorouracil (5-FU) via its efficacious DNA-directed metabolite, FdUMP, while addressing 5-FU's inherent limitations of a narrow therapeutic window, and a short half-life. In some embodiments, FdUMP, a potent suicide inhibitor of thymidylate synthase (TS), is a meagerly generated active metabolite of 5FU, forms a stable micellar complex with the temperature and pH responsive PBC as disclosed herein to form a polymeric micelle complex. The polymeric micelle complex not only targets delivery of FdUMP to the acidic tumor cells rather than normal cells, but also stabilizes FdUMP in its micellar form while in circulation. Furthermore, by directly delivering the more potent FdUMP to the tumor site and circumventing toxicity related to catabolic and RNA-directing metabolites of 5FU, the therapeutic window is expected to significantly increase. Additionally, the prolonged plasma exposures will eliminate the management of frequent IV infusions (8-46 hour), and will increase patient quality of life.

In some embodiments, the ionic agent is cyclic guanosine monophosphate-adenosine monophosphate (cGAMP).

In some embodiments, the ionic agent is a nucleic acid. The nucleic acid can be DNA or RNA. For example, the ionic agent can be mRNA.

In some embodiments, two ionic agents are complexed with the polymeric micelle of the present disclosure. For example, both FdUMP and cGAMP can be complexed with the polymeric micelle.

Polymeric Micelle Complexes

In one aspect, provided herein is a composition comprising a polymeric micelle complex comprising:
i) a plurality of block copolymers, wherein each block copolymer comprises at least a pentablock represented by formula (I) of—[A]-[B]—[C]-[D]-[E]—,
   wherein the repeating units of blocks [A] and [E] each independently comprise a pendant moiety carrying a first charge, and
   wherein blocks [B], [C] and [D] are independently poly(alkylene oxide);
wherein the plurality of pentablock copolymers are arranged into a polymeric micelle with an interior hydrophobic core and an exterior hydrophilic layer; and
ii) one or more ionic agents comprising a first ionic agent, wherein the first ionic agent carries a second charge that is opposite to the first charge of the pendant moiety,
   wherein the first ionic agent complexes with at least a portion of the pendant moieties in the polymeric micelle.

The one or more ionic agents in the polymeric micelle complex described herein can be present at a molar ratio of ionic agent:block copolymer ranging from about of 0.01 to 99:99:0.01. In some embodiments, the ionic agent is present at a molar ratio of ionic agent:block copolymer ranging from about 0.01:1 to about 1:0.01. In some embodiments, the one or more ionic agents are present at a molar ratio of ionic agent:block copolymer ranging from about 0.01:1 to about 1:1. In some embodiments, the one or more ionic agents are present at a molar ratio of ionic agent:block copolymer of more than or about 0.01:1, more than or about 0.011:1, more than or about 0.0125:1, more than or about 0.015:1, more than or about 0.02:1, more than or about 0.05:1, more than or about 0.1:1, more than or about 0.15:1, more than or about 0.25:1, more than or about 0.3:1, more than or about 0.4:1, more than or about 0.5:1, more than or about 0.6:1, more than or about 0.7:1, more than or about 0.8:1, or more than or about 0.9:1.

As a person with ordinary skill in the art would understand that each block copolymer and ionic agent could carry more than one charges depending on functional groups each has. In some embodiments, the ionic agent in the polymeric micelle complex described herein is present in such an amount that the number of the functional group bearing a first charge in the block copolymer is about 2 times or higher than the number of the functional group bearing the opposite charge in the ionic agent. In some embodiments, the ionic agent is present in such an amount that the molar ratio of the functional group bearing a first charge in the block copolymer/the functional group bearing the opposite charge in the ionic agent ranges from about 1:1 to about 100:1. In some embodiments, the ionic agent is present in such an amount that the molar ratio of the functional group bearing a first charge in the block copolymer/the functional group bearing the opposite charge in the ionic agent is more than or about 2:1, more than or about 3:1, more than or about 4:1, more than or about 5:1, more than or about 10:1, more than or about 20:1, more than or about 30:1, more than or about 40:1, more than or about 50:1, more than or about 60:1, more than or about 70:1, more than or about 80:1, or more than or about 90:1.

In some embodiments, the block copolymer carries a positive charge and the one or more ionic agents carry a negative charge. In some embodiments, the block copolymer bearing amine groups carries a positive charge and the one or more ionic agents bearing phosphate groups carry a negative charge.

In some embodiments, the block copolymer bears positively charged amine groups and the one or more ionic agent bear one or more negatively charged phosphate groups, wherein the molar ratio of the positively charged amine groups to the negatively charged phosphate groups (e.g., a molar ratio corresponding to N:P ratios as demonstrated in examples herein) is about 20:1, about 30:1, about 50:1, about 80:1 or about 100:1. In some embodiments, the molar ratio of the positively charged amine groups to the negatively charged phosphate groups ranges from about 10:1 to about 20:1, about 15:1 to about 25:1, about 20:1 to about 30:1, about 25:1 to about 35:1, about 30:1 to about 40:1, about 40:1 to about 50:1, about 45:1 to about 55:1, about 50:1 to about 60:1, about 70:1 to about 80:1, about 75:1 to about 85:1, about 80:1 to about 90:1, about 85:1 to about 95:1, about 90:1 to about 100:1, about 95:1 to about 105:1, or about 100:1 to 110:1. In some embodiments, the molar ratio of the positively charged amine groups to the negatively charged phosphate groups ranges from about 10:1 to about 30:1, about 20:1 to about 40:1, about 40:1 to about 60:1, about 70:1 to about 90:1, or about 90:1 to about 110:1.

In some embodiments, the ionic agent is FdUMP, wherein the molar ratio of the positively charged amine groups to the negatively charged phosphate groups is about 20:1, or ranges from about 15:1 to about 25:1 or about 10:1 to about 30:1. In some embodiments, the ionic agent is mRNA, wherein the molar ratio of the positively charged amine groups to the negatively charged phosphate groups is about 30:1, or ranges from about 25:1 to about 35:1 or about 20:1 to about 40:1. In some embodiments, the ionic agent is cGAMP, wherein the molar ratio of the positively charged amine groups to the negatively charged phosphate groups is about 80:1, or ranges from about 75:1 to about 85:1 or about 70:1 to about 90:1. In some embodiments, the one or more ionic agents are FdUMP and cGAMP, wherein the molar ratio of the positively charged amine groups to the total negatively charged phosphate groups is about 100:1 or ranges from about 95:1 to about 105:1 or about 90:1 to about 110:1. In some embodiments, the one or more ionic agents are mRNA and cGAMP, wherein the molar ratio of the positively charged amine groups to the total negatively charged phosphate groups is about 50:1 or ranges from about 45:1 to about 55:1 or about 40:1 to about 60:1.

In accordance with the present application, the complexation between the block copolymer and one or more ionic agents to form a polymeric micelle complex can be modulated by multiple factors during the complexation process. The complexation process may depend on factors such as molecular structure, size, charge, functional groups, and structural conformation. Ionic interactions, alone or in combination with electrostatic interaction, can be modulated by a combination of hydrophobic interactions, hydrogen bonding, structural conformations, pH of the surrounding milieu thus perturbing the formation of stable micellar complexes. To form polymeric micelle complexes in accordance with the present application, the ionic interactions can be modulated by the pKa or the charge on the block copolymer, the pKa or the perturbed pKa values of the ionic agents, structural features, hydrogen bonding and processing condition that allow both the block copolymer and the ionic agents to ionize. In some embodiments, the ionic agent bears one or more phosphate groups, the pKa of which are appropriate for complexation.

Polymeric micelle complexes can further comprise one or more secondary agents. The use of a secondary agent in preparations of polymeric micelle complex with one or more ionic agents, such as one or more small molecule drugs can increase the efficiency of said drugs.

In some embodiments, the secondary agent is a therapeutic or diagnostic agent, such as a dye (e.g., an imaging dye) and a nucleic acid (e.g., DNA and RNA). For example, indocyanine green (ICG) can be used as an imaging dye in the polymeric micelle complex. In some embodiments, the secondary agent is a hydrophobic agent with a solubility less than 10 μg/ml. In some embodiments, the secondary agent is a hydrophobic agent with a solubility greater than 10 ng/ml. In some embodiments, the secondary agent is complexed to the polymeric micelle via hydrophobic interaction.

In some embodiments, the secondary agent is present in the polymeric micelle complex in the amount of up to about 0.5 weight percent, about 1 weight percent, about 5 weight percent, about 10 weight percent, or about 20 weight percent.

### Pharmaceutical Compositions

Compositions of the present disclosure, such as a polymeric micelle complex containing block copolymers complexed with one or more ionic agents, can be incorporated into a variety of formulations for therapeutic use (e.g., by administration) or in the manufacture of a medicament (e.g., for delivering one or more ionic agents of the present disclosure to a subject in need thereof and/or cell interior of a subject in need thereof and/or for treating or preventing a disease or disorder such as cancer in a subject in need thereof) by combining the composition with appropriate carriers (including, for example, pharmaceutically acceptable carriers or diluents), and may be formulated, for example, into preparations in liquid, aerosolized, semisolid, or powder forms.

In some embodiments, carriers include pharmaceutically acceptable carriers, excipients, or stabilizers that are nontoxic to the cell or subject being exposed thereto at the dosages and concentrations employed. Often the physiologically acceptable carrier is an aqueous pH buffered solution. Suitable physiologically acceptable carriers include, for example, buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®. In some embodiments, the polymeric micelle complex described herein is formulated in a buffer in the pH range of 4.5-8.0, 5.0-8.0, or 5.5-7.5.

Suitable formulations include, for example, solutions, injections, inhalants, microspheres, aerosols, gels, ointments, creams, lotions, powders, dry vesicular powders, tablets, and capsules. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Such diluents include, for example, distilled water, buffered water, physiological saline. PBS. Ringer's solution, dextrose solution, and Hank's solution. A pharmaceutical composition or formulation of the present disclosure can further include, for example, other carriers or non-toxic, nontherapeutic, nonimmunogenic stabilizers, and excipients. The compositions can also include additional substances to approximate physiological conditions, such as pH adjusting and buffering agents, toxicity adjusting agents, wetting agents and detergents. A pharmaceutical composition of the present disclosure can also include any of a variety of stabilizing agents, such as an antioxidant for example.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration (e.g. intrathecal, intramuscular (IM), subcutaneous (SC) and intravenous (IV)), include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In some embodiments, the polymeric micelle complex described herein are formulated for parenteral administration.

Pharmaceutical compositions of the present disclosure containing a composition containing a polymeric micelle complex of the present disclosure may be used (e.g., administered to a subject in need of treatment with a carbohydrate of the present disclosure, such as a human individual) in accord with known methods, such as oral administration, intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes. In some embodiments, compositions and formulations of the present disclosure are useful for subcutaneous (SC), intravenous (IV) or intrathecal administration.

Dosages and desired concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989. pp. 42-46.

For in vivo administration of any of the compositions of the present disclosure containing a polymeric micelle complex, normal dosage amounts may vary from 10 ng/kg up to 100 mg/kg of a subject's body weight per day.

Administration of a composition of the present disclosure containing a polymeric micelle complex can be continuous or intermittent, depending, for example, on the recipient's physiological condition, whether the purpose of the administration is therapeutic or prophylactic, and other factors known to skilled practitioners.

It is within the scope of the present disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

Thus, in some variations, the compositions provided herein may be chronically or intermittently administered to a subject (including, for example, a human) in need thereof. In certain variations, chronic administration is administration of the medicament(s) in a continuous as opposed to acute mode, so as to maintain the initial therapeutic effect (activity) for an extended period of time. In certain variations, intermittent administration is treatment that is not consecutively done without interruption, but rather is cyclic in nature.

### Therapeutic Uses

The present disclosure provides compositions containing a polymeric micelle complex that are capable of delivering one or more ionic agents into the interior of a cell. These compositions are useful for delivering any ionic agent of the present disclosure to a subject in need of such agent.

In some embodiments, the subject is a mammal, such as a human, domestic animal, such as a feline or canine subject, farm animal (e.g., bovine, equine, caprine, ovine, and porcine subject), wild animal (whether in the wild or in a zoological garden), research animal, such as mouse, rat, rabbit, goat, sheep, pig, dog, and cat, and birds. In one embodiment, the subject is a human.

A subject of this disclosure may have any type of cancer. Examples of cancer can include, but are not limited to, adrenal cancer, anal cancer, bile duct cancer, bladder cancer, cancer of the blood, bone cancer, a brain tumor, breast cancer, cancer of the cardiovascular system, cervical cancer, colon cancer, cancer of the digestive system, cancer of the endocrine system, endometrial cancer, esophageal cancer, eye cancer, gallbladder cancer, a gastrointestinal tumor, kidney cancer, laryngeal cancer, leukemia, liver cancer, lung cancer, lymphoma, mesothelioma, cancer of the muscular system, myelodysplastic syndrome, myeloma, nasal cavity cancer, nasopharyngeal cancer, cancer of the nervous system, cancer of the lymphatic system, oral cancer, oropharyngeal cancer, ovarian cancer, pancreatic cancer, penile cancer, pituitary tumors, prostate cancer, cancer of the reproductive system, cancer of the respiratory system, a sarcoma, salivary gland cancer, skeletal system cancer, skin cancer, small intestine cancer, stomach cancer, testicular cancer, thymus cancer, thyroid cancer, bladder cancer, or vaginal cancer. The term ‘lymphoma’ may refer to any type of lymphoma including B-cell lymphoma (e.g., diffuse large B-cell lymphoma, follicular lymphoma, small lymphocytic lymphoma, mantle cell lymphoma, marginal zone B-cell lymphoma, Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). In some embodiments, compositions and formulations containing a polymeric micelle complex as described herein are useful in treating colorectal cancer (CRC), gastrointestinal (GI) cancers, breast cancer, prostate cancer, and head & neck cancer. In some embodiments, compositions and formulations containing a polymeric micelle complex as described herein are useful in treating CRC.

Examples of cancer include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

In some embodiments, compositions and formulations containing a polymeric micelle complex as described herein are useful in treating a liver disease, for example, inflammation in liver. Without wishing to be bound by any theory, it is believed that the compositions and formulations as described herein can accumulate in liver and facilitates efficient payload release in the organ. Any suitable ionic agents known in the art may be used in accordance with the present application. Suitable ionic agents that may be used include, but are not limited to, nonsteroidal antiinflammatory drugs (NSAIDs) or derivatives/metabolites thereof. Exemplary NSAIDs are ibuprofen, naproxen, acemetacin, azaproprazone, fenbufen, feprazone, floctafenine, flufenamic acid, nimesulide, pirprofen, and tiaprofenic acid.

In some embodiments, “treatment” or “treating” includes an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

In some embodiments, “prevention” or “preventing” includes any treatment of a disease or condition that causes the clinical symptoms of the disease or condition not to develop. Compounds may, in some embodiments, be administered to a subject (including a human) who is at risk or has a family history of the disease or condition.

In some variations, an “effective amount” is at least an amount effective, at dosages and for periods of time necessary, to achieve the desired therapeutic or prophylactic result. An effective amount can be provided in one or more administrations.

In some variations, a “therapeutically effective amount” is at least the minimum concentration required to effect a measurable improvement of a particular disease, disorder, or condition, such as a congenital disorder of glycosylation. A therapeutically effective amount herein may vary according to factors such as the disease state, age, sex, and weight of the subject, and the ability of the lipid compositions of the present disclosure to elicit a desired response in the subject. A therapeutically effective amount is also one in which any toxic or detrimental effects of the lipid compositions of the present disclosure are outweighed by the therapeutically beneficial effects.

In one aspect, provided herein is a method for delivering one or more ionic agents to a subject in need thereof. In some embodiments, the method comprises administering to the subject any of the compositions described herein.

In another aspect, provided herein is a method for delivering one or more ionic agents to a cell interior of a subject in need thereof. In some embodiments, the method comprises administering to the subject any of the compositions described herein. In some embodiments, at least a portion of the administered composition traverses the cell plasma membrane to deliver the ionic agent to the cell interior.

In another aspect, provided herein is a method for treating cancer and/or other diseases such as liver diseases, in a subject in need thereof. In some embodiments, the method comprises administering to the subject any of the compositions described herein.

### Articles of Manufacture and Kits

The present disclosure also provides articles of manufacture and/or kits containing a composition of the present disclosure containing a polymeric micelle complex. Articles of manufacture and/or kits of the present disclosure may include one or more containers comprising a purified composition of the present disclosure. Suitable containers may include, for example, bottles, vials, syringes, and IV solution bags. The containers may be formed from a variety of materials such as glass or plastic. In some embodiments, the articles of manufacture and/or kits further include instructions for use in accordance with any of the methods of the present disclosure. In some embodiments, these instructions comprise a description of administration of the composition containing a polymeric micelle complex to deliver the carbohydrate to a subject in need thereof, to deliver one or more ionic agents to a cell interior of a subject in need thereof, or to treat cancer to a subject in need thereof, according to any of the methods of the present disclosure.

The instructions generally include information as to dosage, dosing schedule, and route of administration for the intended treatment. The containers may be unit doses, bulk packages (e.g., multi-dose packages) or sub-unit doses. Instructions supplied in the articles of manufacture and/or kits of the present disclosure are typically written instructions on a label or package insert (e.g., a paper sheet included in the article of manufacture and/or kit), but machine-readable instructions (e.g., instructions carried on a magnetic or optical storage disk) are also acceptable.

The label or package insert indicates that the composition is used for delivering one or more ionic agents (e.g., FdUMP) and/or treating cancer (e.g., CRC). Instructions may be provided for practicing any of the methods described herein.

The articles of manufacture and/or kits of the present disclosure may be in suitable packaging. Suitable packaging includes, for example, vials, bottles, jars, and flexible packaging (e.g., sealed Mylar or plastic bags). Also contemplated are packages for use in combination with a specific device, such as an inhaler, nasal administration device (e.g., an atomizer) or an infusion device such as a minipump. An article of manufacture and/or kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is one or more ionic agents (e.g., FdUMP) capable of treating cancer (e.g., CRC) and/or improving one or more symptoms thereof. The container may further comprise a second active agent.

Articles of manufacture and/or kits may optionally provide additional components such as buffers and interpretive information. Normally, the article of manufacture and/or kit comprises a container and a label or package insert(s) on or associated with the container.

### EXAMPLES

Articles of manufacture and/or kits may optionally provide additional components such as buffers and interpretive information. Normally, the article of manufacture and/or kit comprises a container and a label or package insert(s) on or associated with the container.

Articles of manufacture and/or kits may optionally provide additional components such as buffers and interpretive information. Normally, the article of manufacture and/or kit comprises a container and a label or package insert(s) on or associated with the container.

The following Examples are merely illustrative and is not meant to limit any aspects of the present disclosure in any way.

In the following Examples, the Pentablock copolymer micelles are made of a Pluronic F127 (poly(ethyleneoxide)-block-poly(propyleneoxide)-block poly(ethyleneoxide) (PEO—PPO-PEO) and pH-responsive cationic (poly(2-diethylaminoethyl methacrylate) (PDEAEM) as the end blocks. The amphiphilic Pluronic F127 blocks enhance cellular uptake, while the protonatable tertiary amine groups of PDEAEM facilitate endosomal escape and efficient payload release in the cytoplasm, providing dose-sparing effect. Depending on the PBC concentrations, the formulation can be a liquid micelle solution or a semi-solid thermo-reversible gel.

Exemplary ionic agents that are suitable for use in accordance with the present application are listed in Table 1 below.

| | FdUMP | mRNA | cGAMP |
|---|---|---|---|
| Charge | Anionic | Anionic | Anionic |
| (pH 7.4) | | | |
| Polarity | Polar | Polar | Less polar than |
| | | | FdUMP or mRNA |
| Size | Small | Large | Comparatively |
| | | | smaller/compact |
| pKa | (tri-protic | (tri-protic | (tri-protic |
| | phosphate) 2.15, | phosphate) 2.15, | phosphate) 2,15, |
| | 7.20 and 12.33 | 7.20 and 12.33 | 7.20 and 12.33 |
| Peturbed pKa | 6-6.5 | 5-7 | Greater than 6.5 |
| (physiological | | | |
| pH) | | | |

### Abbreviations

“PBC” corresponds to pentablock copolymer.

“FdUMP” corresponds to 5-Fluoro-2′-deoxyuridine-5′-O-monophosphate.

“ICG” corresponds to indocyanine green.

“PBS” corresponds to phosphate buffered saline.

“cGAMP” corresponds to 2′, 3′ cyclic guanosine monophosphate-adenosine monophosphate.

### Example 1

As described in some embodiments, inherent limitations of fluorouracil (5-FU) was addressed by the design of a novel formulation of FdUMP. The polymeric micelle complexes as exemplified here leverage proven efficacy of 5FU via FdUMP and proven capabilities of the functionalized pentablock copolymer (PBC) for tumor targeting. The polymeric micelle complexes are designed to not only exploit small intracellular pH differences between the tumor cells and normal cells, but also protect FdUMP while in circulation. The polymeric micelle complexes improve upon the proven efficacy of 5-FU, with an increased therapeutic window and a reduced toxicity caused by non-specific targeting and harmful metabolites.

### Preparation of Polymeric Micelle Complex for Intracellular Delivery of FdUMP and ICG

In this Example. FdUMP was electrostatically complexed with the PBC at N/P ratio (N:positively charged amine groups in PBC; and P:negatively charged phosphate groups in FdUMP) of up to 100:1. The complexation was conducted at room temperature by either protonating the polymer initially or incubating the PBC with the drug for 30 min in phosphate buffered saline (PBS) at pH 7.4. The ratio of Polymer:drug_N/P is important along with the process of protonation of the polymer, complexation with the drugs and neutralization to enable complexing. The following formulations were prepared and tested for performance using different methods as outlined in Table 2. Examples of preparation for formulations (A and A1), a non-optimal formulation (B), a formulation of free drug without any PBC (C) and a formulation of ICG within the PBC (D) are listed below.

TABLE 2
Formulations Prepared and Performance Test

| Formulations | Anionic drug | N/P ratio | Performance Test |
|---|---|---|---|
| A | FdUMP | 80 | PK in rats |
| A1 | FdUMP | 100 | Dialysis followed |
| | | | by HPLC assay |
| B | FdUMP | 20 | PK in rats |
| C | FdUMP | 0 | PK in rats |
| D | ICG | — | PK in rats |

Formulation A:Procedure for the Preparation of 5 ml of the FdUMP Formulation (0.25 mg/ml FdUMP, 20 mg/ml Polymer, N:P Ratio of 80:1):
1. Dissolved 100 mgs of the polymer in 1.25 ml of 10 mM PBS (pH 7.4).
2. Added 125 μl of 1N HCl and mix by vortexing.
3. Added 625 μl FdUMP (2 mg/ml solution in water) solution to the protonated polymer solution.
4. Stirred for 10 mins.
5. Neutralized with 1.25 ml of 200 mM PBS (pH7.4).
6. Mixed by vortexing or stirring until clear and homogenous for 10-15 mins.
7. QS to volume with water to 5 ml
8. Filtered through 0.2 micron filter.
   Formulation A1:Procedure for the Preparation of 5 ml of the FdUMP Formulation (0.25 mg/ml FdUMP. 20 mg/ml Polymer. Polymer:Drug N:P Ratio 80:1):
1. Dissolved 100 mgs of the polymer in 1.25 ml of 10 mM PBS (pH 7.4).
2. Added 125 μl of 1N HCl and mix by vortexing.
3. Added 625 μl FdUMP (2 mg/ml solution in water) solution to the protonated polymer solution.
4. Stirred for 10 mins.
5. Neutralized with 1.25 ml of 200 mM PBS (pH7.4).
6. Mixed by vortexing or stirring until clear and homogenous for 10-15 mins.
7. QS to volume with water to 5 ml
8. Filtered through 0.2 micron filter.
   Formulation B:Procedure for the Preparation of 5 ml of the FdUMP Formulation (1 mg/ml FdUMP, 20 mg/ml Polymer, Polymer:Drug N:P Ratio 10:1):
1. Dissolved 100 mgs of the polymer in 1.25 ml of 10 mM PBS (pH 7.4).
2. Mixed by vortexing.
3. Added 1250 μl FdUMP (4 mg/ml solution in water) solution to the polymer solution.
4. Stirred for 10 mins.
5. Added 1.25 ml of 200 mM PBS (pH7.4).
6. Mixed by vortexing or stirring until clear and homogenous for 10-15 mins.
7. QS to volume with water to 5 ml.
8. Filtered through 0.2 micron filter.
   Formulation C:Procedure for the Preparation of 5 ml of the FdUMP Formulation (0.3 mg/ml FdUMP. 0 mg/ml Polymer. Polvmer:Drug N:P Ratio 0:100):
1. Dissolved 1.2 mgs of the FdUMP in 5 ml of 10 mM PBS (pH 7.4).
2. Mixed by vortexing or stirring until clear and homogenous.
3. Filtered through 0.2 micron
   Formulation D:Procedure for the Preparation of 5 ml of the ICG Formulation (0.25 mg/ml ICG, 20 mg/ml Polymer. Polvmer:Drug N:P Ratio 80:1)
1. Dissolved 100 mgs of the polymer in 1.25 ml of 10 mM PBS (pH 7.4).
2. Mixed by vortexing.
3. Added 625 μl of ICG (2 mg/ml solution in water) solution to the polymer solution.
4. Stirred for 10 mins.
5. Added 1.25 ml of 10 mM PBS (pH7.4).
6. Mixed by vortexing or stirring until clear and homogenous.
7. QS to volume with water to 5 ml.
8. Filtered through 0.2 micron filter.

### Characterizations of Formulation A1

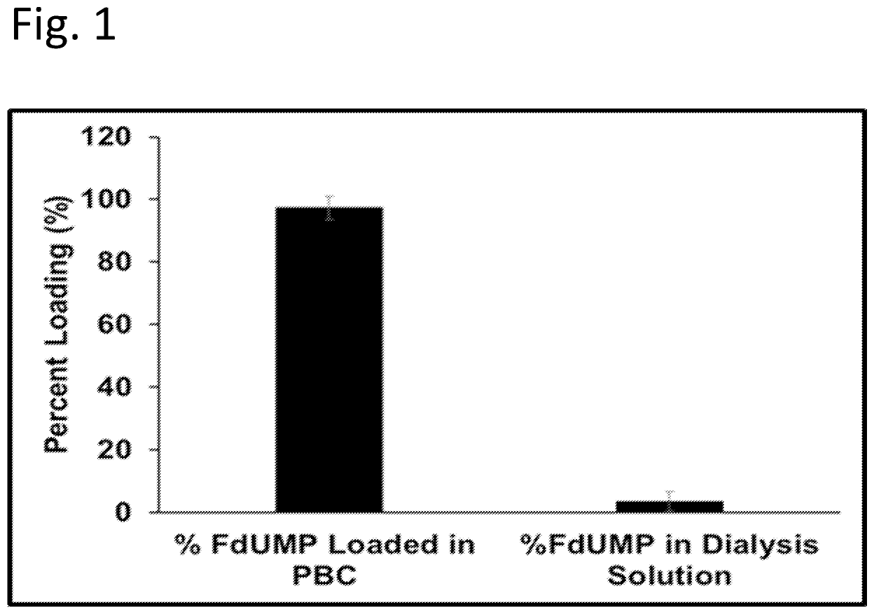

In this Example, Formulation A1 prepared as described above was characterized. The complexation was conducted at room temperature for 30 min in PBS buffer at pH 7.4. FdUMP (5 μg in 200 μL) is mixed with PBC (200 μg/200 μL). After that, the PBC/FdUMP complex (400 μL) was injected into a dialysis bag (MWCO: 3500 Da) and placed in 20 mL of PBS buffer and left for 2 days. At the end of the dialysis, the PBC/FdUMP complex (400 μL) in dialysis bag and dialysate solution was collected for the detection of FdUMP amount. FdUMP was detected in HPLC using C18 column and a UV detector (˜270 nm). The Water:Acetonitrile:Methanol (60:20:20) was used as mobile phase. No significant amounts of FdUMP were observed in the dialysate solution, however, all of the initial amount of FdUMP remained as a micellar complex in the dialysis bag. This indicated almost 100% of complexation. All the dilutions and controls were taken into consideration during the measurements. PBC alone and FdUMP alone at initial amounts were used as control for HPLC measurements. FIG. 1 outlines the results of the dialysate and the PBC:FdUMP complex at N/P ratio of 100:1.

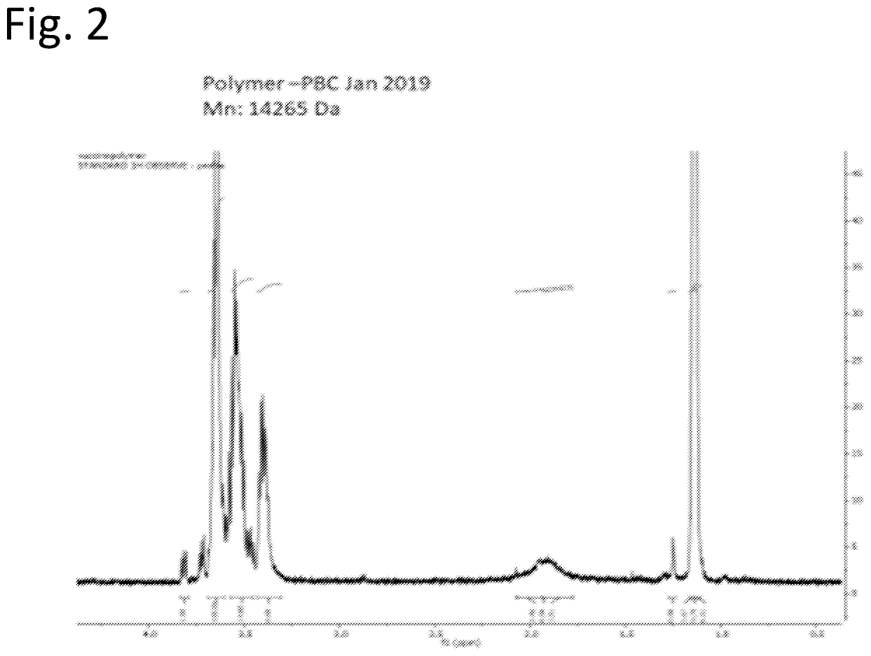

NMR Characterization of the PBC was conducted prior to complexation. FIG. 2 is the NMR data confirming structure of the polymer.

### In Vivo Pharmacokinetic Study in Rats

A 42-day tolerance study in rats (n=3) dosed IV once weekly (5 doses) with the PBC as described above at 200 mg/Kg showed it was safe. No clinical signs of toxicity were observed.

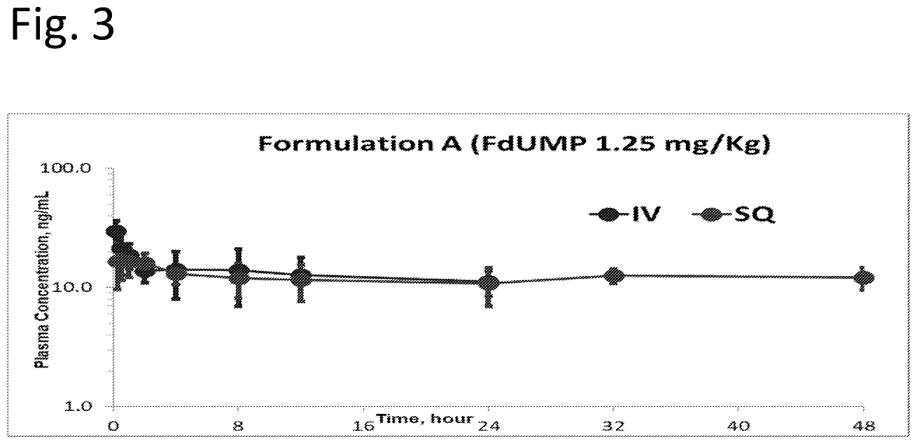

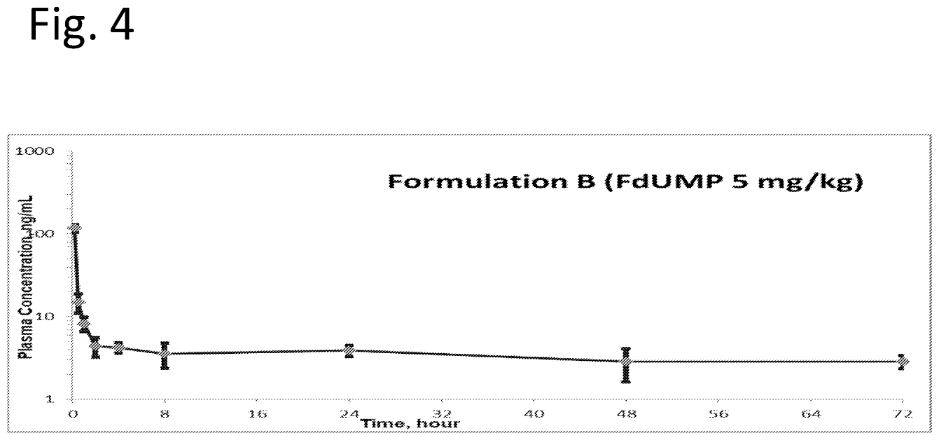

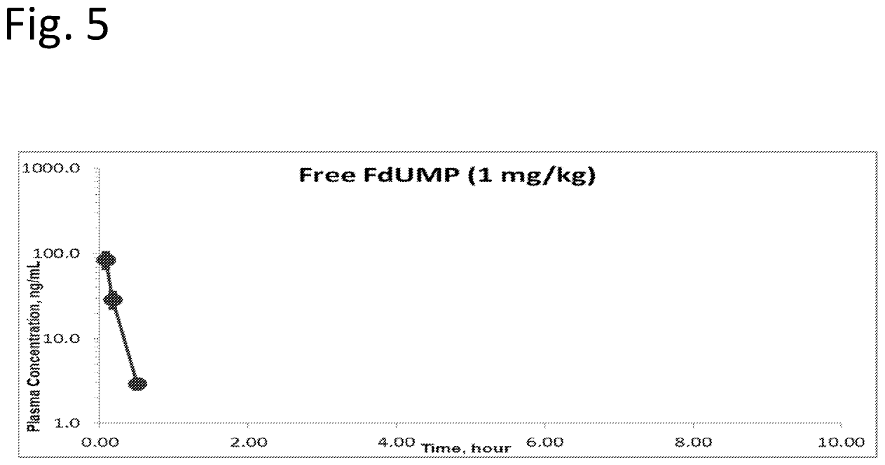

Furthermore, in vivo pharmacokinetic study of formulations prepared as described above was conducted. Sprague Dawley rats (n=3) were dosed Formulations A, B and C intravenously (IV) at 5 ml/Kg dose volume through the tail vein. Blood sampling for rats dosed with Formulations A and B was done at pre-determined times: 10 mins, 30 mins, 1 hr, 4 hr, 8 hr, 24 hr, 48 hr post-dose. Blood sampling for rats dosed with Formulations C was done at pre-determined times: 0, 5 mins, 10 mins. 30 mins. 1 hr. 2 hr. 4 hr. 8 hr and 24 hr post-dose. Plasma was processed from blood samples by centrifugation at 2-8° C. at 5000 rpm for 5 mins and frozen at −80° C. until submitted for LC-MS analysis for FdUMP content. Formulation A was also dosed subcutaneously (SC) in rats. Formulation A (1.25 mg/Kg) had the optimal complexation of FdUMP with the polymer as seen by the prolonged half (t½=53 hours by IV and 116 hours (FIG. 3). Formulation B, dosed at 4 times higher dose (5 mg/Kg) had prolonged exposure and half-life of FdUMP (FIG. 4). However, lot of the drug exposure was within the first 10 minutes post-dosing and the area under the curve (AUC) was lower than that of Formulation A even though it was dosed 4 times higher. Results indicate non-optimal and partial complexation of FdUMP with the polymer. Formulation C, which had no polymer and only the free FdUMP showed no prolongation of exposure with a half-life of <10 mins (FIG. 5).

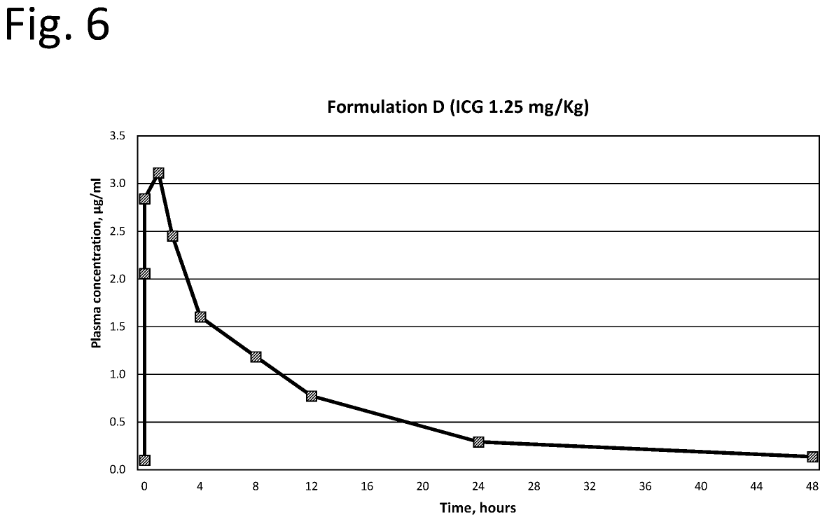

Formulation D (250 μg/ml), which had the polymer complexed with ICG was also tested for ICG levels (FIG. 6).

### Example 2

In other embodiments, the polymeric micelle complexes can be used for delivery of nucleic acids. As exemplified here, polymeric micelle complexes can be prepared with nucleic acids, including messenger RNAs (mRNAs). Polymeric micelle complexes with mRNAs can deliver these mRNAs preferentially into the interior of a cell, such as that of a tumor, where the mRNAs can be translated into proteins. In some embodiments, the delivered mRNAs can encode for fluorescent markers such as green fluorescent protein (GFP). In other embodiments, the delivered mRNAs can encode a protein of therapeutic use.

Preparation of Formulations of PBC with mRNA

In this example, cyanine-tagged EGFP mRNA was complexed with the PBC at N/P (N:positively charged amine groups in PBC; and P:negatively charged in mRNA) ratios of 10:1, 30:1, and 50:1. The complexation was conducted at room temperature by incubating the PBC with the mRNA for 30 minutes on a shaker. Formulations of PBC with mRNA (E, E1, E2, H. and H1), a formulation of free mRNA without any PBC (F), and a formulation of PBC without any mRNA (G) were prepared.

To prepare the formulations listed above, solutions of PBC and EGFP mRNA were first prepared. The PBC solution was prepared at a concentration of 20 mg/mil of PBC in RNAse-free 1×TBE buffer (Tris-borate-EDTA buffer). TBE is a buffer solution made up of Tris base, boric acid and EDTA. The EGFP mRNA was prepared at a concentration of 0.2 μg/μl by diluting 10 μl cyanine-tagged EGFP mRNA (1 μg/μl) with 40 μl 1×TBE buffer. The EGFP solution was mixed by vortexing and kept on ice. For complexation, cyanine-tagged EGFP mRNA (0.2 μg/μl) was titrated with PBC. 1×TBE was then added to the titration. After addition of 1×TBE, the mix was vortexed, then incubated to allow for complexation. All incubations were performed on a shaker at room temperature for 30 minutes.

Formulations of PBC with mRNA were prepared at N:P ratios of 10:1 (E), 30:1 (E1), and 50:1 (E2). For Formulation E, 2 μl of (20 μg/μl PBC) were mixed with 10 μl mRNA (0.2 μg/μl). Then, 8 μl of 1×TBE buffer were added before mixing by vortex. For preparing Formulation E1, 6 μl of (20 μg/p1 PBC). 10 μl mRNA (0.2 μg/μl), and 4 μl of 1×TBE buffer were used. For preparing Formulation E2, 10 μl of (20 μg/μl PBC) and 10 μl mRNA (0.2 μg/μl) were used, without addition of 1×TBE.

The control mRNA formulation (F) was prepared by mixing 10 μl of 1×TBE buffer with 10 μl of EGFP mRNA (0.2 μg/μl). The control PBC formulation (G) was prepared by mixing 10 μl of 1×TBE buffer with 10 μl of PBC (20 μg/μl). As for formulations E, E1, and E2, the control formulations were incubated at room temperature for 30 minutes.

### Analysis by Gel Electrophoresis

Formulation E, E1, E2, F, and G were prepared as described above and analyzed by agarose gel electrophoresis. A 1% agarose gel was prepared and 20 μl of each formulation was loaded at the center of the gel. The gel was run for 30 minutes after addition of 5 μl of loading dye. The gel was then visualized on a fluorescence gel imager (LI-COR Odyssey).

Mg. 7 shows a fluorescence image of the agarose gel result. The observed red fluorescence corresponds to signal from the cyanine-tagged EGFP. Cyanine is a red fluorescent tag. The gel image showed red fluorescence and captured the complexation of mRNA with PBC at different N:P ratios In the gel, PBC-RNA complexes in Formulations E, E1, and E2 moved towards the negative electrode, while free mRNA in Formulation F moved towards the positive electrode. No fluorescence signal is detected in Formulation G.

### In Vivo Expression of EGFP Protein in Tumor

Two separate formulations (H and H1) of mRNA complexed with PBC were prepared and injected intra-tumorally in mice bearing xenograft tumors generated from a HCT116 colo-rectal cancer cell line.

Two formulations of PBC with mRNA were prepared for intra-tumoral injection. For preparation of formulation H, 20 mgs of PBC were dissolved in 0.5 ml of water to prepare a 40 mg/mL PBC solution. The PBC solution was vortexed until a homogenous solution was obtained. The pH of the PBC solution was not adjusted. 50 μl of PBC solution (40 mg/ml) were added 100 μl EGFP mRNA (1 μg/μl) and mixed by vortexing. For complexation, the preparation was incubated on a shaker for 30 mins at room temperature. Formulation H1 was prepared as Formulation H, except that PBC was dissolved in 10× TBE to prepare the PBC solution. Both Formulations H and H1 had an N:P ratio of 10:1.

### In Vivo Intra-Tumoral Injection

Two intra-tumoral injection experiments were conducted with 3 mice each. Formulation H was tested in the first experiment, while Formulation H1 was tested in the second. The procedure is summarized below.

To generate mice bearing xenograft tumors, HCT-116 cells were inoculated into the left flank of 3 nude mice. When tumor volume reached approximately 300 mm³, 50 μl of either Formulation H or H1 were intra-tumorally injected. Tumors were harvested 48 hours-post-injection and weighed. Each tumor was then homogenized in 500 μl of PBS and freeze-thawed 4 times to lyse the tumor cells. After the lysis, the homogenate was spun at 3,000 rpm for 5 minutes. A 100 μl supernatant was used to measure GFP activity (Excitation at 485 nm, Emission at 520 nm). Each measurement was performed in triplicate. Table 3 summarizes the results of these experiments.

TABLE 3
Results of the GFP fluorescence measurement in the mice tumors:

| | | | | | | | | | | | GFP |
|---|---|---|---|---|---|---|---|---|---|---|---|
| | | | | | | | | | | | Fluores- |
| | | | | | | | | | | | cence Δ |
| Mouse | | Tumor | Water | GFP readout (100 ul/well), | | | | GFP readout (100 ul/well), | | | Sample |
| Tumor | | weight | added | raw data | | | | subtracted background | | | vs |
| # | Treatment | (mg) | (ul) | n = 1 | n = 2 | n = 3 | Average | n = 1 | n = 2 | n = 3 | control |
| 1 | Control | 261 | 500 | 10412 | 10391 | 10380 | 10394 | 17.7 | −3.3 | −14.3 | 0.0 |
| 2 | Test | 204 | 500 | 10474 | 10594 | 10512 | 10527 | 79.7 | 199.7 | 117.7 | 132 |
| 3 | Test | 343 | 500 | 11486 | 11635 | 11620 | 11580 | 1091.7 | 1240.7 | 1225.6 | 1186 |

* formulation leakage from the tumor resulted in mis-dosing and insufficient volume dosed

No difference was observed between the fluorescence readout of the control and treated samples in tumors injected with Formulation H in the first experiment. The results indicated that the experiment was unsuccessful and the mRNA did not express the GFP protein in the tumors. The pH of formulation H was not adjusted with TBE buffer and may have been acidic.

In addition, tumor in mouse 3 exhibited fluorescence specific to the GFP protein indicating expression of the GFP protein. The expression of GFP indicated successful complexation and delivery of EGFP mRNA to the tumor. Formulation H1 successfully protected the mRNA until it was delivered into the tumor.

### Example 3

Cyclic dinucleotide (CDN) agonists of stimulator of interferon genes (STING) are a promising class of immunotherapeutic agents that activate innate immunity to increase tumor immunogenicity. In some embodiments, the polymeric micelle complexes can be used for delivery of cyclic dinucleotides (CDNs). As exemplified here, polymeric micelle complexes can be prepared with an endogenous CDN ligand for stimulator of interferon genes (STING), 2′, 3′ cyclic guanosine monophosphate-adenosine monophosphate (cGAMP). The stabilized polymeric micellar complex could enhance therapeutic efficacy of cGAMP alone or in combination with other drugs such as FdUMP.

The structure of cGAMP is shown below.

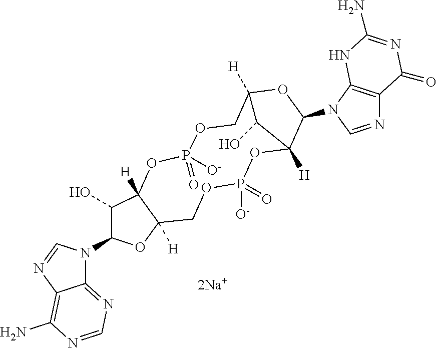 [SMILES: Nc1nc(=O)c2ncn([C@@H]3O[C@@H]4COP(=O)([O-])O[C@H]5[C@@H](O)[C@H](n6cnc7c(N)ncnc76)O[C@@H]5COP(=O)([O-])O[C@@H]3[C@@H]4O)c2[nH]1]

Preparation of Formulations of PBC with cGAMP or Combination cGAMP and UMP

In this Example, cGAMP was complexed with the PBC at N/P ratio (N:positively charged amine groups in PBC; and P:negatively charged phosphate groups in cGAMP) of up to 100:1. The complexation was conducted at room temperature by incubating the PBC with the drug for 30 min in phosphate buffered saline (PBS) at pH 7.4 or TBE buffer (pH 8). The ratio of Polymer:drug_N/P is important along with the pH of the milieu (pH 7.4 or pH 8). The following formulations were prepared and tested for performance using different methods as outlined in

Table 4. Preparation for exemplary formulations are described below. PGP.46,T1,M

TABLE 4
Formulations Prepared and Performance Test

| | Ionic | | |
|---|---|---|---|
| Formulations | agents(s) | N/P ratio | Performance Test |
| X1 | cGAMP | 100 | Dialysis followed by HPLC assay |
| X2 | cGAMP | 80 | Dialysis followed by HPLC assay |
| Y | cGAMP | 40 | Dialysis followed by HPLC assay |
| Y1 | cGAMP | 10 | Dialysis followed by HPLC assay |
| Z | cGAMP, | 100 | Dialysis followed by HPLC assay |
| | FdUMP | | |
| Z1 | cGAMP, | 25 | Dialysis followed by HPLC assay |
| | FdUMP | | |
| Z2 | cGAMP, | 50 | Dialysis followed by HPLC assay |
| | mRNA | | |

Formulation X:Procedure for the preparation of 2 ml of the cGAMP polymeric formulation (0.25 mg/mi cGAMP. 50 mg/ml Polymer. N:P ratio of 100:1): The starting volumes of the polymer, cGAMP or FdUMP solutions can be reduced as long as the N:P ratio is maintained.
The solution can be diluted post complexation.
1. Dissolved 100 mgs of the polymer in 1.00 ml 1×TBE buffer (pH 8).
2. Added 1000 μl cGAMP (0.5 mg/ml solution in 1×TBE buffer) solution to the polymer solution.
3. Stirred for 30 mins to 1 hour at room temperature.
4. Mixed by vortexing or stirring for an additional 2-5 minutes
5. Filtered through 0.2 micron filter.
   Formulation X1:Procedure for the preparation of 2 ml of the cGAMP polymeric formulation (0.25 mg/ml cGAMP, 40 mg/ml Polymer, N:P ratio of 80:1): The starting volumes of the polymer, cGAMP or FdUMP solutions can be reduced as long as the N:P ratio is maintained.
   The solution can be diluted post complexation.
1. Dissolved 80 mgs of the polymer in 1.00 ml of 1×TBE buffer (pH 8).
2. Added 1000 μl cGAMP (0.5 mg/ml solution in 1× TBE buffer) solution to the polymer solution.
3. Stirred for 30 mins to 1 hour at room temperature.
4. Mixed by vortexing or stirring for an additional 2-5 minutes.
5. Filtered through 0.2 micron filter.
   Formulation Y:Procedure for the preparation of 2 ml of the cGAMP polymeric formulation (0.25 mg/ml cGAMP, 20 mg/ml Polymer, N:P ratio of 40:1): The starting volumes of the polymer, cGAMP or FdUMP solutions can be reduced as long as the N:P ratio is maintained.
   The solution can be diluted post complexation.
1. Dissolved 40 mgs of the polymer in 1.00 ml of 1× TBE buffer (pH 8).
2. Added 1000 μl cGAMP (0.5 mg/ml solution in 1× TBE buffer) solution to the polymer solution.
3. Stirred for 30 mins to 1 hour at room temperature.
4. Mixed by vortexing or stirring for an additional 2-5 minutes.
5. Filtered through 0.2 micron filter.
   Formulation Y1:Procedure for the preparation of 2 ml of the cGAMP polymeric formulation (0.25 mg/ml cGAMP. 5 mg/ml Polymer. N:P ratio of 10:1): The starting volumes of the polymer, cGAMP or FdUMP solutions can be reduced as long as the N:P ratio is maintained.
   The solution can be diluted post complexation.
1. Dissolved 10 mgs of the polymer in 1.00 ml of 1× TBE buffer (pH 8).
2. Added 1000 μl cGAMP (0.5 mg/ml solution in 1× TBE buffer) solution to the polymer solution.
3. Stirred for 30 mins to 1 hour at room temperature.
4. Mixed by vortexing or stirring for an additional 2-5 minutes.
5. Filtered through 0.2 micron filter.
   Formulation Z:Procedure for the preparation of 2 ml of combination FdUMP and cGAMP polymeric formulation (2.5 m/ml FdUMP. 0.25 mg/ml cGAMP. 50 mg/ml Polymer, N:P ratio of 100:1): The starting volumes of the polymer, cGAMP or FdUMP solutions can be reduced as long as the N:P ratio is maintained. The solution can be diluted post complexation
1. Dissolved 100 mgs of the polymer in 1.00 ml of 1× TBE buffer (pH 8).
2. Added 500 μl cGAMP (1.0 mg/ml solution in 1× TBE buffer) solution to the polymer solution.
3. Mixed by vortexing for 3-5 mins.
4. Added 500 μl of FdUMP (5 mg/ml in DI water) to the polymeric solution (step 3)
5. Stirred for 30 mins to 1 hour at room temperature.
6. Mixed by vortexing for an additional 2-5 minutes.
7. Filtered through 0.2 micron filter.
   Formulation Z1:Procedure for the preparation of 2 ml of combination FdUMP and cGAMP polymeric formulation (2.5 mg/ml FdUMP, 0.25 mg/ml cGAMP, 12.5 mg/ml Polymer, N:P ratio of 25:1): The starting volumes of the polymer, cGAMP or FdUMP solutions can be reduced as long as the N:P ratio is maintained. The solution can be diluted post complexation.
1. Dissolved 25 mgs of the polymer in 1.00 ml of 1×TBE buffer (pH 8).
2. Added 500 μl cGAMP (1.0 mg/ml solution in 1×TBE buffer) solution to the polymer solution.
3. Mixed by vortexing for 3-5 mins.
4. Added 500 μl of FdUMP (5 mg/ml in DI water) to the polymeric solution (step 3)
5. Stirred for 30 mins to 1 hour at room temperature.
6. Mixed by vortexing for an additional 2-5 minutes.
7. Filtered through 0.2 micron filter.
   Formulation Z2:Procedure for the preparation of combination mRNA and cGAMP polymeric formulation (0.125 mg/ml cGAMP, 0.05 μg/ml mRNA, 30 mg/ml Polymer, N:P ratio of 50:1): The starting volumes of the polymer, cGAMP or mRNA solutions can be reduced as long as the N:P ratio is maintained. The solution can be diluted post complexation.
1. Hundred μl of mRNA complexed micellar Formulation E2, was prepared as described in Example 2 and mixed with 100 μl of cGAMP micellar Formulation X.
2. The mixture was vortexed for 5 minutes and incubated for 20 minutes at room temperature to generate Formulation Z2.
3. The formulation is stored at 2-8° C. until analyzed or used.

### Characterization of Formulations

After complexation PBC/cGAMP or PBC/cGAMP/FdUMP complex (400 μL) was injected into a dialysis bag (MWCO: 3500 Da) and μlaced in 20 mL of PBS buffer and left for 2 days. At the end of the dialysis, the PBC/cGAMP or PBC/cGAMP/FdUMP complex (400 μL) in dialysis bag and dialysate solution was collected for the detection of either cGAMP or FdUMP amounts. FdUMP was detected by HPLC using C18 column and a multi-wavelength UV detector (˜270 nm or 254 nm) using a 1% to 5% CH₃CN in 0.1 M NH₄HCO3 gradient or Water:Acetonitrile:Methanol (60:20:20) was as mobile phase. No significant amounts of cGAMP were observed in X, X1, Y, Z, Z1 formulations. No significant levels of FdUMP were observed in the dialysate solutions in formulation Z and Z1. No significant levels of cGAMP were observed in the dialysate solution in formulations Z2. All of the initial amount of FdUMP and/or cGAMP remained complexed/micellized with the polymer in the dialysis bag. This indicated almost 100% of complexation. All the dilutions and controls were taken into consideration during the measurements. PBC alone and FdUMP alone at initial amounts were used as control for HPLC measurements. About 8% of uncomplexed cGAMP was observed in the dialysate from formulation Y1 indicating incomplete complexation at the ratio of N:P used.

### Example 4

### Evaluation of Key Properties

Further evaluation of key properties of formulations in accordance with the present application are ongoing including (a) Metabolic Stability of polymeric micelle complexes in Human. Rat, and Dog Liver Microsomes (b) Stability of polymeric micelle complexes in rat, dog and human blood and plasma (c) Demonstrate cellular uptake of polymeric micelle complexes in HT-29 cells. Summary these studies is outlined in Table 5.

TABLE 5
Characterization Key properties of polymeric micelle complexes

| | |
|---|---|
| Description | Data Analysis |

Metabolic Stability in mouse, rat, dog and human liver microsomes

| | |
|---|---|
| Metabolic stability of polymeric | The extent of metabolism = |
| micelle complexes are evaluated | disappearance of polymeric |
| using mouse, rat, dog and human | micelle complexes, compared |
| hepatocytes to predict intrinsic | to the 0-min control. Initial |
| clearance. polymeric micelle | rates are calculated for |
| complexes (1 μM and 0.5 million | polymeric micelle complexes |
| cells/mL hepatocytes) are incubated | concentration and used to |
| for 0, 60, 120, and 180 minutes | determine t1/2 values and |
| at 37° C. with hepatocytes in 96-well | subsequently, the intrinsic |
| micro-titer plates. At each time point | clearance, CLint = ke*V = |
| 200 μL of quench solution (100% | (0.693)(1/t1/2 (min))(mL |
| acetonitrile with 0.1% formic acid) | incubation/million cells). |
| with internal standard is transferred | polymeric micelle |
| to each well. Plates are sealed and | complexes = 1 μM: Positive |
| centrifuged at 4° C. for 15 minutes | control: midazolam and/ |
| at 4000 rpm. The supernatant is | or naloxone [Hepatocyte] = |
| transferred to fresh plates for | 0.5 million cells/mL |
| LC/MS/MS analysis. | Time: 0, 60, and 180 min; |
| | Temperature = 37° C. |

Micellar Stability in human blood and plasma

| | |
|---|---|
| Stability of polymeric micelle | Recovery of polymeric micelle |
| complexes in human blood and | complexes in human blood and |
| plasma are evaluated by | plasma is compared to the control |
| incubating polymeric micelle | polymeric micelle complexes |
| complexes (1 μM) in human | incubated in PBS under same |
| blood and plasma for 4 hours | conditions. Quantitation are |
| at 37° C.. Supernatants are analyzed | done by LC/MS/MS. |

Cellular uptake of Polymeric Micelle Complex HT-29 Cancer Cells

| | |
|---|---|
| Polymeric micelle complexes are | Confocal Microscopy imaging |
| prepared using the Alexa Fluor | are done for the qualitative |
| 488 dye-attached PB copolymers. | evaluation of cellular uptake, |
| HT-29 cells are plated at a density | intracellular distribution and |
| of 2 × 105 cells per well in cell | endosomal escape of |
| culture petri dishes and incubated | polymeric micelle complexes |
| for 24 h at 37° C. Then, the dye | in HT-29 cells for live cell |
| labeled polymeric micelle | imaging, the lysosomes |
| complexed will be added | stained with Lysotracker Red |
| to the wells and incubated | and nucleus will Hoechst. |
| with the cells for 24 h. | |

### Example 5

### Efficacy in HT-29, HT-116 and Other Mouse Xenograft Models

Efficacy (PD) of polymeric micelle complexes in HT-29, HT-116 and other mouse Xenograft model is being evaluated, at doses of 10, 30 and 50 mg/Kg doses based on a mouse efficacious dose (MED) projection of 30 mg/Kg from published data and our plasma exposure data in rats, allometrically scaled in mice. A companion PK study of polymeric micelle complexes dosed IV at the same 3 doses are conducted in CD-1 mice. Mice (n=3/time point) are sampled via saphenous vein bleeding. Each mouse is bled 4 times including a terminal bleed. Samples are processed for polymeric micelle complexes quantitation as outlined in Example 1. The xenograft study design is based on published studies with some modifications. A comparator 5FU arm at MED dose is added. The colons of CD-1 mice (n=6/group), are orthotopically implanted with luciferase labeled HT-29 cells to establish xenograft tumors. Ten days post-implantation, mice are imaged with an IVIS spectrum imager (Perkin Elmer, USA) after a luciferin intraperitoneal (IP) injection (D-luciferin 100 μl at 15 mg/ml in PBS). Based on luciferase expression, mice are randomized into four groups (n=6 animals/group). During the course of the treatment, the tumor growth is monitored non-invasively with the imager on days 0, 1, 8, 15 and 22 (study endpoint) of treatment. Mice are sacrificed at the study endpoint and tumors harvested and weighed. Half of each primary tumor is flash frozen for TS expression studies and the other half is fixed in 10% buffered formalin for immune-histochemistry (IHC)(optional) and protein analysis. Polymeric micelle complexes are quantitated in terminal plasma samples using LC/MS/MS. The summary design is outlined in Table 6.

TABLE 6
Summary Design-Efficacy (PD) in HT-29 and other
Xenograft model and PK in CD-1 mice

| | | IV Dosage | Tumor | Terminal |
|---|---|---|---|---|
| | Dose | Schedule | imaging | Day |
| Group | (mg/Kg) | (days) | (days) | (day 22) |
| Vehicle | 0 | PD study: | 0, 3, 8, | PD: (HT-29 Xenograft |
| *5FU | 65 | 0, 3, 8, 15 | 15, 22 | Mice) terminal blood |
| polymeric | 50 | PK study: 0 | | sampling for plasma |
| micelle | | | | exposure; tumor |
| complexes | | | | harvesting; tumor size |
| polymeric | 30 | | | measurement; ½ of |
| micelle | | | | tumor is flash frozen |
| complexes | | | | (TS expression): other |
| polymeric | 10 | | | ½ in 10% formalin for |
| micelle | | | | Immunohistochemistry |
| complexes | | | | (IHC) |
| | | | | PK: (CD-1) mice at all |
| | | | | doses, n = 3: PK |
| | | | | sampling time: 10 min, |
| | | | | 0.5 h, 1 h, 2 h, 4 h, 8 h, |
| | | | | 24 h, 48 h, 72 |

Efficacy: Mice/group = 6/group-Xenograft model;
PK: Mice/group = 3/time point;
*efficacious 5FU dose

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

1. A polymeric micelle complex comprising:
i) a plurality of block copolymers, wherein each block copolymer comprises at least a pentablock represented by formula (I) of
—[A]-[B]-[C]-[D]-[E]—,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer; and
ii) one or more ionic agents comprising a first ionic agent, wherein the first ionic agent carries a second charge that is opposite to the first charge of the pendant moiety,
wherein the first ionic agent complexes with at least a portion of the pendant moieties in the polymeric micelle.

2. The polymeric micelle complex of claim 1, wherein the blocks [A] and [E] each independently have a structure: 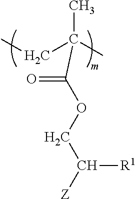
wherein
R¹ is selected from the group consisting of a hydrogen and a C₁₋₆ alkyl group; 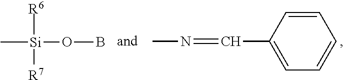
Z is selected from the group consisting of NR²R³, P(OR⁴)₃, SR⁵
wherein R² and R³ are independently H, C₁₋₆ alkyl, or 1-mer to 28-mer oligonucleotide in which one or more of its natural phosphate backbone linkages are replaced with triazole linkages, or R² and R³ together with the nitrogen form a cyclic amine;
R⁴ is C₁₋₆ alkyl;
R⁵ is tri(C₁₋₆alkyl) silyl; and
B is C1-6 alkyl;
and
m is an integer ranging from 1 to 5000.

3. The polymeric micelle complex of claim 2, wherein R¹ is H.

4. The polymeric micelle complex of claim 2, wherein Z is NR²R³ and at least one of R² and R³ is 1-mer to 28-mer oligonucleotide in which one or more of its natural phosphate backbone linkages are replaced with triazole linkages.

5. The polymeric micelle complex of claim 2, wherein Z is NR²R³ and R² and R³ together with the nitrogen form a cyclic amine.

6. The polymeric micelle complex of claim 5, wherein the cyclic amine is selected from the group consisting of pyrrolidine, piperidine, morpholine, and piperazine.

7. The polymeric micelle complex of claim 2, wherein Z is NR²R³ and R² and R³ are the same C₁₋₆ alkyl.

8. The polymeric micelle complex of claim 7, wherein R² and R³ are both ethyl.

9. The polymeric micelle complex of claim 2, wherein m is 13.

10. The polymeric micelle complex of claim 1, wherein the blocks [A] and [E] are pH-responsive.

11. The polymeric micelle complex of claim 1, wherein the pendant moieties of blocks [A] and [E] are cationic.

12. The polymeric micelle complex of claim 1, wherein the alkylene oxide unit of the blocks [B] and [D] are unsubstituted and unbranched, and the alkylene oxide unit of the block [C] is substituted or branched.

13. The polymeric micelle complex of claim 1, wherein the blocks [B] and [D] are the same 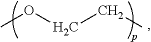
wherein p is an integer ranging from 30 to 20,000.

14. The polymeric micelle complex of 13, wherein the ratio of m:p is in the range of 0.1 to 1.

15. The polymeric micelle complex of 14, wherein m is about 13 and p is about 100.

16. The polymeric micelle complex of claim 1, wherein the block [C] is 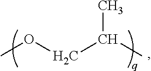
wherein q is an integer ranging from 1 to 20,000.

17. The polymeric micelle complex of 16, wherein the ratio of p:q is in the range of 10 to 1.

18. The polymeric micelle complex of 17, wherein p is about 100 and q is about 65.

19. The polymeric micelle complex of claim 1, wherein the first ionic agent is complexed to at least a portion of the pendant moieties via at least ionic interaction.

20. The polymeric micelle complex of claim 1, wherein the first ionic agent is Floxuridine, Fluorouracil, Azathioprine, Thiopurines, Fludarabine, Gemcitabine, Cytarabine, Methotrexate, Pemetrexed or Paracetamol, or a derivative or metabolite thereof.

21. The polymeric micelle complex of claim 20, wherein the first ionic agent is 5-fluoro-2′-deoxyuridine-5′-O-monophosphate (FdUMP).

22. The polymeric micelle complex of claim 1, wherein the first ionic agent is cyclic guanosine monophosphate-adenosine monophosphate (cGAMP).

23. The polymeric micelle complex of claim 1, wherein the first ionic agent is a nucleic acid.

24. The polymeric micelle complex of claim 23, wherein the first nucleic acid is mRNA.

25. The polymeric micelle complex of claim 1, wherein the first ionic agent is present at a molar ratio of ionic agent:block copolymer ranging from about 0.01:1 to about 1:0.01 and/or in such an amount that the number of the functional group bearing a first charge in the block copolymer is at least 2 times or higher than the number of the functional group bearing the opposite charge in the first ionic agent.

26. The polymeric micelle complex of claim 1, further comprising a second ionic agent, wherein the second ionic agent carries the second charge that is opposite to the first charge of the pendant moiety and wherein the second ionic agent complexes with at least a portion of the pendant moieties in the polymeric micelle.

27. The polymeric micelle complex of claim 26, wherein the first and second ionic agents are 5-fluoro-2′-deoxyuridine-5′-O-monophosphate (FdUMP) and cyclic guanosine monophosphate-adenosine monophosphate (cGAMP), or mRNA and cyclic guanosine monophosphate-adenosine monophosphate (cGAMP).

28. The polymeric micelle complex of claim 1, wherein the first and second ionic agent is present at a total molar ratio of ionic agent:block copolymer ranging from about 0.01:1 to about 1:0.01 and/or in such an amount that the number of the functional group bearing a first charge in the block copolymer is at least 2 times or higher than the number of the functional group bearing the opposite charge in the first and second ionic agent.

29. The polymeric micelle complex of claim 1, further comprising further comprising a secondary agent.

30. The polymeric micelle complex of claim 29, wherein the secondary agent is a hydrophobic agent with a solubility greater than 10 μg/ml or greater than 10 ng/ml.

31. The polymeric micelle complex of claim 29, wherein the secondary agent is a therapeutic or diagnostic agent.

32. The polymeric micelle complex of claim 31, wherein the secondary agent is an imaging dye or a nucleic acid.

33. A pharmaceutical composition comprising the polymeric micelle complex of claim 1, and a pharmaceutically acceptable carrier.

34. The pharmaceutical composition of claim 33, wherein the polymeric micelle complex formulated in a buffer in the pH range of 4.5-8.0 and/or other pharmaceutically acceptable solvents for parenteral administration.

35. A method for delivering one or more ionic agents to a human in need thereof, comprising administering to the human the polymeric micelle complex of claim 1.

36. A method for delivering one or more ionic agents to a cell interior of a subject in need thereof, comprising administering to the subject the polymeric micelle complex of claim 1, wherein at least a portion of the administered composition traverses the cell plasma membrane to deliver the ionic agent to the cell interior.

37. A method for treating a disease in a human in need thereof, comprising administering to the human the polymeric micelle complex of claim 1.

38-44. (canceled)

45. A kit comprising: the polymeric micelle complex of claim 1.

46. (canceled)

47. (canceled)

48. A method of preparing the polymeric micelle complex of claim 1.
